(19) United States
(12) Patent Application Publication
(10) Pub. No.: US 2026/0076915 A1
(43) Pub. Date: Mar. 19, 2026

(54) MODIFIED RELEASE FORMULATION COMPRISING CAPSAICINOIDS AND A PROCESS

(71) Applicant: NUTRIVENTIA LIMITED, Andheri (East) Mumbai (IN)

(72) Inventors: Rajat SHAH, Mumbai (IN); Vishal SHAH, Mumbai (IN); Shajahan ABDUL, Madurai (IN)

(21) Appl. No.: 19/110,077

(22) PCT Filed: Dec. 9, 2022
(86) PCT No.: PCT/IB2022/061975

§ 371 Date: Mar. 9, 2025

(30) Foreign Application Priority Data
Oct. 14, 2022 (IN) 202221058793

(51) Int. Cl. A61K 9/50; A23L 33/105; A23P 10/30; A61K 36/81

(52) U.S. Cl. CPC A61K 9/5036; A23L 33/105; A23P 10/30; A61K 9/5015; A61K 9/5026; A61K 36/81

(57) ABSTRACT

The present invention relates to a modified release capsaicinoids formulation comprising of active core, at least one emulsifier and at least one gastro-resistant pH dependent polymer. The active core may be coated with at least one release modifying agent and optionally the outermost coating. The invention as described herein also relates to the solvent-free process, wherein emulsion comprising capsaicinoids is entrapped in at least one gastroresistant pH dependent polymer and deposited on non-pareil seeds to get active core, which may be further coated using at least one release modifying agent to get the beadlets. The formulation releases not more than 10% by weight of capsaicinoids in stomach within 2 hours, followed by release of not less than 75% of capsaicinoids in the intestine within 1 hour. The modified release formulation does not exhibit the alcohol dose dumping effect in stomach and it is safe for oral administration to the consumers.

### CROSS-REFERENCE TO RELATED APPLICATIONS

This application is the U.S. national stage of International Patent Application No. PCT/IB2022/061975, filed Dec. 9, 2022, which claims priority to Indian Application No. 202221058793, filed Oct. 14, 2022.

### FIELD OF THE INVENTION

The present invention relates to a modified release formulation comprising capsaicinoids and a process for preparation thereof. The formulation may be comprised of an active core comprised of capsaicinoids, at least one emulsifier, at least one gastro-resistant pH dependent polymer and at least one processing aid, acceptable in pharmaceutical, nutraceutical and food industry. The active core is coated with at least one release modifying agent and optionally further covered by the outermost coating, but the formulation is free of use of organic solvents. The formulation may be comprised of 0.5 to 20% of capsaicinoids, 0.5 to 10% of emulsifiers, 0.5 to 10% by weight of at least one gastro-resistant pH dependent polymer and 15 to 50% of at least one release modifying agent or the combination thereof. The formulation may be comprised of combination of emulsifiers obtained from natural, synthetic and semi-synthetic sources, having varying HLB values. The invention as described herein also relates to the solvent-free process for preparation of the modified release formulation, wherein emulsion comprising capsaicinoids is entrapped in at least one gastro-resistant polymeric excipient and deposited on non-pareil seeds to get the active core, which may be further coated using at least one release modifying agent to get the capsaicinoids beadlets, which may be optionally further coated with protective polymers. The process for preparation is cost effective, environment friendly and employs industrially available equipment. The beadlet formulation is non-sticky, free flowing and the particle size may range from 100 to 1000 microns. The formulation as described herein, releases not more than 10% by weight of capsaicinoids in stomach within 2 hours, followed by not less than 75% by weight of capsaicinoids in the intestine within 1 hour, after oral administration. The formulation can be converted in suitable dosage forms for convenient oral administration to the consumers.

The formulation is robust for effect of alcohol and it does not exhibit alcohol induced dose dumping in the stomach. The formulation, as described herein can be administered safely to the subjects as a sports nutrition, for enhancing the exercise capacity and endurance and for weight management.

### Prior Art and Problem to be Solved

Peppers, inclusive of chili peppers, red peppers and paprika, are one of the most widely consumed food, especially in the form of a popular culinary spice for food throughout the world since 7000BC. After being introduced from the Americas, chili pepper is also incorporated prominently in diets of various communities and cultures globally with a long history of flavouring, colouring, preserving food as well as nutritional content and medication. Peppers belong to the genus Capsicum. C. annuum, C. frutescens, C. chinense, C. pubescenes, and C. baccatum are grown domestically or commercially. Of these Capsicum annuum is grown most extensively.

Pepper plants are shallow-rooted and lack a taproot, which are notorious for their sensitivity to moisture stress at flowering and fruit setting. Peppers are an excellent source of phytochemicals, such as anthocyanins, vitamins, phenolic acids, flavonoids, carotenoids, and capsaicinoids. Various studies have demonstrated the benefits of bioactive compounds of peppers in vitro and in vivo. Capsaicinoids are the constituents in pepper that are responsible for pungency. The degree of pungency is characterized in terms of Scoville heat units. (SHU) measured based on the concentrations of capsaicinoid compounds within the fruit. SHU scale measures the number of times the extract is diluted to make pungency undetectable in sugar water. Physiologically, capsaicinoids are synthesized by the condensation of vanillyl amine produced by the phenylpropanoid pathway and a branched-chain fatty acid produced by the catabolism of amino acids. Within the placental tissues of the developing pepper fruit, capsaicinoids are synthesized 20 to 30 days after pod formation and continue to accumulate as the fruit matures.

In chilli pepper, more than 200 active constituents have been identified and some of its active constituents play multiple roles in the whole body. The active biochemical constituents of peppers provide many nutritional and health benefits that include antioxidant, anti-inflammatory, and antimicrobial activities, anticancer therapy, reduced prevalence of type 2 diabetes and obesity, protection against hypercholesterolemia, and reduced prevalence of atherosclerotic cardiovascular diseases. The active constituents are especially useful for reduction in body mass, percentage body fat, waist circumference, and a desirable reduction in levels of critical markers of weight maintenance such as blood glucose, insulin, triacylglycerol, and leptin.

Capsaicinoids, as a major active compound from chili pepper, has been also established for its numerous beneficial roles, including the treatment of pain, inflammation, rheumatoid arthritis, and vasomotor rhinitis. Furthermore, it was postulated that this compound has important implications in the prevention or treatment of neurodegenerative diseases such as Alzheimer's disease. Thus there is an immense potential for nutritionists to incorporate capsicum and its active compounds in various formulations for achieving desired benefits. (Trends in Plant Science-Volume 24, Issue 2, February 2019, Pages 109-120).

Capsaicinoids are well known for their pungency and these cause irritation to the skin and mucous membrane. The pungency is measured by Scoville Heat Units (SHU) and in order to be effective as a dosage form, greater than 10,000-20,000 SHU are required to be administered to the consumer. Accordingly, the starting material in manufacturing systems are found at heat levels typically as high as 250,000-10,00,000 SHU (i.e., 25-50 times “hotter” than what is to be “consumed” by the end user). It is very difficult to convert the capsaicinoids into dosage forms such as tablets/capsules due to their skin irritation properties as the operators working on the granulation or tablet compression machine may not tolerate the intense pungency arising out of the very fine dust particles of capsicum. Further, the release of significant amount of capsaicinoids from the dosage form in the stomach may cause nausea, vomiting, abdominal pain, diarrhoea, a burning sensation and gastric upset including irritating the stomach lines.

Prior search references indicate few efforts for designing the capsicum formulations with sustained, delayed and extended-release profile, based on matrix or coating systems which make use of sustained release hydrophilic matrix or cellulose polymers or layering of the active using organic solvents, which are not environment friendly and safe for the human consumption and also do not ensure complete absorption of the pungent active from intestine.

U.S. Pat. No. 10,668,123 relates to the composition containing capsaicinoids and non-capsaicinoids constituents, such as flavonoids and saponins in specific ratio which is evaluated in individuals with high risk of developing or suffering from cardiometabolic syndrome. The subject under evaluation is administered specific diet wherein 70% of daily calories from carbohydrates or about 32 to 60% of daily calories from fat for a period of eight weeks and has a high risk of developing or suffering from cardiometabolic disease. The patent mainly aims at evaluation of capsaicinoid containing composition for effect on cardiometabolic syndrome in individuals consuming the high calories diet, however it does not show any efforts in optimization of formulation to ensure the desired use.

U.S. Pat. No. 11,382,879 describes use of capsicum composition for improving physical performance. The methods include administering a capsicum composition to the exercising subject, such as walking on a treadmill, in an effective amount to reduce blood lipids and oxidative stress. The disclosure provides evaluation of the composition in experimental animals, when the animal is subjected to very specific exercise activity such as walking on treadmill; however the patent doesn't provide any insight with respect to formulation components, its release profile or any efforts to obtain the said effect in the subjects.

U.S. Pat. No. 10,813,967 relates to delayed, extended and sustained release matrix formulation in which the nutrient core of the active along with cellulosic polymer, starch, sugar, and other excipient is entrapped using enteric coating polymer by extrusion spheronization method. The formulation exhibits minimum release of active constituent in stomach, followed by delayed and extended release of the active in the intestine area. This may result in the incomplete absorption off active from the intestine, resulting in irritation in the colon and rectum due to unabsorbed active from the formulation.

U.S. Pat. No. 9,254,268 describes the oral compositions of lipophilic active compounds wherein polymeric matrix is formed by two or more polymers possessing a hydrophobic-hydrophilic range. The composition forms colloidal nanodispersion upon contact with aqueous media and permits immediate release. The process involves use of water and organic solvent to prepare solution of active and polymer which is further dried to prepare solid composition.

IN254661 relates to the capsicum beadlet composition which is consisting of hydrophilic polymer, selected from hydroxypropyl methyl cellulose, polyvinyl pyrrolidone, starch, hydroxypropyl cellulose and polyethylene glycol. The process for preparation involves use of a combination of polar and non-polar solvents to make dispersion of the active substance. Such formulation may contain residual solvents and therefore may not be a safe choice for administration to the consumers.

The compositions, as described in the prior art, do not address the issue of designing a formula and process, which is safe for both handling and administration to the consumers. None of the prior art references provide a formulation free of organic solvents, safe for consumption to the consumers and also a process which is user friendly, to avoid discomfort to the formulator in the lab and also to the users after oral administration, which ensures complete release and absorption in the intestine.

There exists a need for developing a formulation and process, which addresses the difficulty of handling the pungent actives like capsaicinoids during handling and manufacture. The need also exists for developing the formulation which also facilitates administration of capsaicinoids in effective and high doses in suitable dosage forms, which can avoid exposure of consumer to the pungent smell, odour or other sensory attributes of the active, thus enhancing the compliance for long term use. The formulation also needs to ensure complete release and absorption in the intestine, thus avoiding the discomfort and ill effects due to residual pungent active in the large intestine and the end parts of gastro-intestinal tract.

### Objects of the Invention

The main objective of the invention is to provide a modified release formulation comprising capsaicinoids and a solvent free process for the preparation thereof.

Another important objective of the instant invention is to provide a modified release formulation, comprising of active core comprising capsaicinoids, at least one emulsifier, at least one gastro-resistant pH dependent polymer and at least one processing aid, which may be coated with at least one release modifying agent or the combination thereof, and optionally further coated with the outermost coating.

One more important objective of the invention is to provide a formulation, comprised of about 0.5 to 20% of capsaicinoids, 0.5 to 10% of emulsifiers, 0.5 to 10% of at least one gastro-resistant pH dependent polymer and 15 to 50% of at least one release modifying agent or the combination thereof.

Still one more important objective of this invention is to provide a formulation, comprised of about 10 to 60% by weight of the non-pareil seeds, selected from the material having acidic, basic as well as neutral properties, such as tartaric acid, lactose, microcrystalline cellulose and cellulose derivatives, starch, sucrose, mannitol, sugar, isomalt, xylitol, anhydrous dibasic calcium phosphate, calcium carbonate, silica, and the combination thereof.

Another objective of the present invention is to provide the modified release capsaicinoids formulation, comprised of an emulsifier or the combination of emulsifiers, with varying HLB values, obtained from natural, semi-synthetic and synthetic sources.

Another important objective of the invention is to provide a modified release formulation, comprised of at least one gastro-resistant pH dependent polymer, which entraps capsaicinoids emulsion before deposition on the non-pareil seeds.

One objective of the present invention is to provide the capsaicinoid formulation, which may be comprised of at least one processing aid, acceptable in pharmaceutical, nutraceutical and food industry.

One more important objective of the present invention is to provide a solvent-free process for preparation of the modified release formulation, wherein emulsion comprising capsaicinoids and a combination of emulsifiers is entrapped in at least one gastro-resistant pH dependent polymer and deposited on non-pareil seeds to get active core, which may be further coated using at least one release modifying agent to get the capsaicinoids beadlets.

Still one more objective of the instant invention is to provide a solvent-free process for preparation of modified release formulation, thus making it safe for administration, cost effective and environment friendly

Another objective of the present invention is to provide the formulation in the form of capsaicinoids beadlets, which is non-sticky, free flowing and the particle size of the beadlets may range from 100 to 1000 microns.

Still another objective of the instant invention is to provide a modified release formulation, which exhibits release of not more than 10% by weight of capsaicinoids in stomach within 2 hours after administration, followed by release of not less than 75% by weight of capsaicinoids in the intestine within 1 hour.

One important objective of the invention is to provide the formulation, which is robust for effect of alcohol and it does not exhibit alcohol induced dose dumping effect in the stomach.

Another important objective of the present invention is to provide the modified release formulation comprising capsaicinoid beadlets, which can be converted in suitable compressible dosage form, can be filled in capsules or used in the form of sachets as dry mix for beverage, dry syrups, or may be formulated as liquid syrup, health drink, diet drink, and can also be administered through fruit juices, soft drinks and the like, for convenient administration to the consumers.

The formulation, as described herein can be administered safely to the subjects as a sports nutrition, for enhancing the exercise capacity and endurance and for weight management.

### SUMMARY OF THE INVENTION

The researchers of the present invention have carried out extensive experimentation to optimise the process and selection of excipients in capsaicinoid formulation, to avoid the discomfort during preparation of the formulation and after administration to the consumers. The optimised selection of non-pareil seeds, one or more emulsifiers, at least one gastro-resistant polymer, at least one release modifying agent and at least one processing aid, has resulted in a formulation which releases less than 10% by weight of capsaicinoids in the stomach till 2 hours after administration and exhibits immediate complete release in the intestine within 1 hour.

The formulation eliminates gastric discomfort and facilitates complete intestinal absorption of the active ingredient, thereby minimizing the discomfort caused by residual unabsorbed active ingredient in stomach. The formulation, as described herein may also be further optionally covered by outermost coating, which may facilitate its oral consumption in the delivery form, such as sachets for use as dry mix for beverage, dry syrups, or also in the form of liquid syrup, health drink, diet drink, through fruit juices, soft drinks and the like, to the consumers and may enhance compliance for long term use. The outermost coating may reduce the possibility of exposure of the consumers to the pungent taste or odor, and related sensory characteristics of administration of such formulation, even at sufficiently high dosages.

The modified release formulation, as described herein, may be comprised of combination of emulsifiers, obtained from natural, semi-synthetic and synthetic sources, having varying HLB values. The invention as described herein also relates to the solvent-free process for preparation of the modified release formulation, wherein emulsion comprising capsaicinoids is entrapped in at least one gastro-resistant pH dependent polymer and deposited on non-pareil seeds to get active core, which may be further coated using at least one release modifying agent to get the capsaicinoids beadlets. The formulation as described herein, releases not more than 10% by weight of capsaicinoids in stomach within 2 hours, followed by release of not less than 75% by weight of capsaicinoids in the intestine within 1 hour. The coated beadlet formulation may be further coated with an optional outermost coat. The modified release formulation of the present invention is robust for effect of alcohol, and it does not exhibit alcohol induced dose dumping effect in the stomach. The formulation is prepared by solvent-free process and can be administered safely to the subjects. None of the prior art provides such a formulation comprising capsaicinoids, which is safe and convenient for preparation and administration and robust for effect of alcohol in the stomach.

### BRIEF DESCRIPTION OF THE ACCOMPANYING FIGURES

FIG. 1 is a scanning electron microscopy (SEM) photograph of one embodiment of a composition, which is formed by depositing the emulsion of capsaicionoids entrapped in at least one gastro-resistant pH dependent polymer, coated by at least one release modifying agent, resulting from Formula 5.

FIG. 2 is scanning electron microscopy (SEM) photograph of one embodiment of a composition which is formed by depositing the emulsion of capsaicionoids entrapped in at least one gastro-resistant pH dependent polymer, coated by at least one release modifying agent, resulting from Formula 7.

FIG. 3 is scanning electron microscopy (SEM) photograph of one embodiment of a composition which is formed by depositing the emulsion of capsaicionoids entrapped in at least one gastro-resistant pH dependent polymer, coated by at least one release modifying agent, and optionally applying outermost coating, resulting from Formula 10.

### DETAILED DESCRIPTION OF THE INVENTION

The present invention describes a modified release formulation comprising capsaicinoids, which may be comprised of at least one emulsifier or a combination thereof, at least one pH dependent gastro-resistant polymer and at least one release modifying agent or the combination thereof. The formulation is prepared in the form of non-sticky and free-flowing beadlets, varying in size from 100 to 1000 microns, which can be converted in suitable compressible dosage form, can be filled in capsules or used in the form of sachets for administration in the form of dry-mix for beverage and variety of suitable dosage forms. The invention also relates to the solvent-free process for preparation of beadlet formulation comprising capsaicinoids, which is safe for administration to the consumers.

The formulation may be comprised of capsaicinoids, which is a highly pungent constituent, obtained from chilli concentrate, in the form of dark red capsicum oleoresin, purchased from Kandukur, Telangana. Capsicum is the dried ripe fruit of various Capsicum species (Family-Solanaceae). It contains NLT 0.3% of total capsaicinoids, calculated as the sum of capsaicin, dihydrocapsaicin, nordihydrocapsaicin, nonivamide, decanylvanillinamide, and homocapsaicin; all calculated on the dried basis.

Capsicum Oleoresin is an alcoholic concentrate of the dried ripe fruits of Capsicum. It contains NLT 6.5% of total capsaicinoids, calculated as the sum of capsaicin, dihydrocapsaicin, nordihydrocapsaicin, nonivamide, decanylvanillinamide, and homocapsaicin, all calculated on the anhydrous basis.

Total capsaicinoids content in the capsicum oleoresin is determined by using High Performance Liquid Chromatography equipped with pump, UV detector at 281 nm wavelength and having HPLC column, 4.6-mm×25-cm; end-capped, 5-μm, 150 Å, packing L11, maintained at 30° C. The Mobile phase is a mixture of acetonitrile and diluted phosphoric acid (1 in 1000) (2:3). The standard solutions are 0.2 mg/mL of USP Capsaicin RS in methanol (Solution A) and 0.1 mg/mL of (SP Dihydrocapsaicin RS in methanol (Solution B). The sample solution is 5 mg/mL of Capsicum Oleoresin in methanol after filtration through a filter of 0.2-μm pore size. The 20 μL of both standard and sample solutions are injected with 1 mL/min flow rate to estimate the total content of capsaicinoids in the capsicum oleoresin sample. The capsaicin and dihydrocapsaicin peaks are identified in the Sample solution chromatogram by comparison with the chromatograms of Standard solution A and Standard solution B, respectively. The peaks corresponding to nordihydrocapsaicin, nonivamide, decanylvanillinamide, and homocapsaicin are identified using the approximate relative retention times as 0.89, 0.95, 1.34, 1.40 respectively. The content of total capsaicinoids is calculated as the sum of the percentages of capsaicin, dihydrocapsaicin, nordihydrocapsaicin, nonivamide, decanylvanillinamide, and homocapsaicin.

The active substance may be also available in the form of capsaicin powder. The capsaicin powder may contain more than 90% of total capsaicinoids. The powder form of capsaicin may be dissolved in oily substances before formulating modified release formulation. The oily substances employed may be selected from sunflower oil, palm oil, mustard oil coconut oil, groundnut oil, gingerly oil, and mixture thereof. The oily dissolved capsaicin powder may be comprised of about 80% of total capsaicinoids. The active starting material used within the scope of the present invention may be in the form of capsaicin powder or capsicum oleoresin.

As per main embodiment of the invention, the modified release formulation of the invention, as described herein may be comprised of 0.1 to 50% by weight of capsaicinoids. More preferably, the formulation may be comprised of 0.5 to 20% by weight of total capsaicinoids, synonymously referred to as capsaicinoids, in the present invention. Most preferably, the formulation may be comprised of 1 to 10% by weight of capsaicinoids, as per the present invention.

The modified release formulation, as described herein is comprised of capsaicinoids and at least one emulsifier or the combination thereof, selected from natural, semi-synthetic or synthetic source.

The terminology ‘modified release’ as used herein refers to the release of the active (total capsaicinoids or synonymously capsaicinoids) from a formulation which is different than the generally known immediate, sustained or extended drug release, so that the release of active is avoided or tolerable amount of capsaicinoids is released in stomach over first 2 hours, thus the pungency or irritation to the mucous lining is avoided and there is quick and complete release of active in the intestine. The modified release formulation comprising capsaicinoids, exhibits not more than 10% release of active in the stomach in first 2 hours and the complete release of not less than 75% of the active is achieved within 1 hour, after the formulation enters the intestine.

According to the important embodiment of the invention, the modified release formulation avoids release of capsaicinoids in the stomach for 2 hours after oral administration and facilitates immediate release of capsaicinoids in the alkaline environment of the intestine within 1 hour to ensure complete absorption in the intestine. The formulation, as described herein avoids discomfort in the stomach and the entire gastro-intestinal tract, or any resulting irritation in the large intestine or the rectal area due to unabsorbed or residual capsaicinoids from the formulation. The release profile of the formulation is thus modified to prevent the release of the active ingredient in gastric parts of the digestive system and achieve complete intestinal absorption of the active ingredient in the intestine to eliminate any discomfort or irritation due to pungent characteristic of the active.

The terminology ‘active core’ as referred within the scope of the present invention, may relate to the composition which makes use of non-pareil seeds as a spherical bead, which provides inert substrate for coating, depositing or layering of the active, as described herein, which is comprised of capsaicinoids, at least one emulsifier and at least one gastro-resistant pH dependent polymer or the combination thereof. Therefore, the active core, as described herein, is a non-pareil seed deposited with the active capsaicinoids in the form of emulsion entrapped in at least one gastro-resistant pH dependent polymer.

The terminology ‘emulsifier or emulsifying agent’ as used within the scope of present invention relates to the excipient, which is used as an aid to convert the active into an emulsion and it also stabilizes the emulsion. The emulsifier can be available from natural, synthetic or semi-synthetic source and it can possess varying HLB (hydrophilic-lipophilic balance) value. All emulsifiers work at the oil: water interface and provide a protective barrier around the dispersed droplets. Emulsifiers may also stabilize the emulsion by reducing the interfacial tension of the system. Some emulsifiers may improve stability of emulsion by imparting a charge on the droplet surface thereby reducing the physical contact between the droplets and hence decreasing the potential for coalescence.

Emulsifiers can be also classified based on their chemical structure, mechanism of action or solubility in water. A very useful important technique used for the classification of emulsifiers based on their solubility in water was introduced by Griffin in the form of numerical values, which is called as hydrophilic-lipophilic balance (HLB) which denotes the relative affinity of the emulsifier for oil and water. The Emulsifying agents with HLB values of 3 to 6 are used for w/o emulsions preparations, whereas the emulsifying agents with HLB values of 7 to 20 are used for preparation of o/w emulsions. The type of emulsion is a function of the relative solubility of the emulsifier, therefore the phase in which the emulsifier is more soluble gives rise to the continuous phase.

An emulsion is a two-phase system consisting of at least two immiscible liquids, one of those liquids which is dispersed in the form of small droplets throughout the other, and with the help of an emulsifier. The dispersed liquid is known as the internal or discontinuous phase, whereas the dispersion medium is known as the external or continuous phase. The system having oils, lipids, or lipophilic substances as the dispersed phase, and the water or an aqueous solution in the continuous phase, the system is called an oil-in-water (o/w) emulsion. On the other hand, the system wherein the water or aqueous solutions are dispersed in an oily, fatty or lipophilic medium, then the system is known as water-in-oil (w/o) emulsion.

According to an important embodiment of the present invention, the capsaicinoids may be combined with at least one emulsifier or the combination thereof, to form the emulsion of capsaicinoids, within the scope of this invention. The emulsifier may be selected from natural, synthetic or semi-synthetic source, having varying HLB values.

As per one embodiment of the invention, the emulsifier may be selected from but not limited to potassium laurate, triethanolamine stearate, sodium lauryl sulfate, alkyl polyoxyethylene sulfates, sodium dodecyl sulfate, and Dioctyl sodium sulfosuccinate, Quaternary ammonium compounds such as cetyltrimethyl ammonium bromide, and lauryldimethyl benzylammonium chloride, polyoxyethylene fatty acid derivatives of the sorbitan esters (for example, Tween series), polyoxyethylene fatty alcohol ethers, sorbitan fatty acid esters, polyoxyethylene alkyl ethers, polyoxyethylene sorbitan fatty acid esters, polyoxyethylene polyoxypropylene block copolymers such as poloxamers, polyethylene glycol 400 monostearate, glycerol monostearate, lanolin alcohols, ethoxylated lanolin and the combination thereof.

According to further embodiment of the present invention the modified release formulation may be comprised of at least one emulsifier from synthetic origin, which may be used in the range of 0.5% to 10% w/w of the formulation.

According to one more embodiment of the present invention, the emulsifier obtained from natural source may belong to vegetable or animal origin and may be selected from, but not limited to quillaia concentrate, acacia, tragacanth, alginates, chondrus, xanthan, pectin, gelatin, egg yolk, casein, wool fat, cholesterol, wax, lecithin and the mixture thereof.

As per further embodiment of the present invention, the modified release formulation, may be comprised of an emulsifier obtained from the natural source, which may be used in the range of 0.5 to 10% by weight of the formulation.

As per one more embodiment of the present invention, the emulsifier obtained from semi-synthetic origin may be selected from cellulose derivatives, such as methyl cellulose, carboxymethyl cellulose, sodium carboxymethyl cellulose and may be used in the range of 0.5 to 10% by weight of the formulation.

The modified release formulation, as described herein, may be preferably comprised of a combination of emulsifier, obtained from natural, synthetic or semi-synthetic source, and may be used in the range of 0.5 to 10% by weight of the capsaicionoids formulation.

The formulation may be comprised of at least one emulsifier or a combination thereof, to prepare a stable emulsion of capsaicinoids, as per the process of the invention. The emulsifier/s aid for dispersing the oily active well in water and resulting into a homogenous emulsion of capsaicinoids during the process for preparation of the formulation.

As per the preferred embodiment of the present invention, a combination of quillaia concentrate and glyceryl monostearate may be used during the formation of stable emulsion comprising capsaicinoids. The emulsifier quillaia (synonymously called as quillaja) is obtained as a concentrate from wild Quillaia Saponaria Molina and it appears as a amber coloured liquid with slight odour. It is used as a liquid emulsifier in a range of clear beverages, including popular functional and flavoured waters.

Glycerol monostearate, commonly known as GMS, is a monoglyceride commonly used as an emulsifier in foods. It takes the form of a white, odorless, and sweet-tasting flaky powder that is hygroscopic. Chemically it is the glycerol ester of stearic acid. GMS is a food additive used as a thickening, emulsifying, anticaking, and preservative agent; an emulsifying agent for oils, waxes, and solvents; a outermost coating for hygroscopic powders; a solidifier and control release agent in pharmaceuticals; and a resin lubricant. As per the scope of present invention, it is used as emulsifier in the active core in the amount ranging from 0.5 to 5% by weight of the formulation. It is also used as a lubricant in the coating composition in the amount ranging from 1 to 20%, considering both the functional coating and optional outermost coating.

As per one embodiment of the present invention, active may be heated with an emulsifier, such as glyceryl monostearate (HLB 3.8) to get the oil phase, which can be mixed with water phase to get water in oil emulsion. This emulsion may be further added to aqueous solution of emulsifier, such as quillaia concentrate (HLB 13.5) to get oil in water emulsion. In some embodiments, the modified release formulation may be comprised of a combination of emulsifiers in weight percentage (w/w) at a range of at or about 0.5 to 10%, wherein each or individual emulsifier may range in weight percentage varying from 0.5 to 5%.

The terminology ‘at least one gastro-resistant pH dependent polymer’ as used within the scope of the present invention relates to the excipients, which are water miscible, water dispersible or water soluble; and can be used to prepare a matrix wherein the active can be embedded well, so that the matrix of gastro-resistant polymer comprising the active, will avoid release of active in the acidic pH environment of the stomach, after oral administration of the formulation. These polymers exhibit pH dependent property; thus protect the embedded active and will not allow its release or exposure to the acidic environment of gastrointestinal tract, however the polymers will dissolve or disintegrate to release the embedded active from the matrix, only in the alkaline environment of the intestine. The gastro-resistant pH dependent polymer as used herein, may enhance the release of active in the intestine, resulting in release of more than 70% of the capsaicinoids within one hour in the intestine for quick or immediate absorption. Therefore, it avoids the release of active in stomach where the released pungent active from core may cause irritation to delicate mucous lining. Thus, by virtue of its pH dependent release characteristics, the release modifying agent modifies release of the pungent active and thus avoids its release in stomach, thus protecting gastric mucosa from possible irritation which may be caused due to release of pungent capsaicinoids in the stomach.

According to important embodiment of the present invention, the gastro-resistant pH dependent polymer, as described herein, is used in the core of the formulation, exhibits good miscibility or dispersibility with water and it forms homogeneous dispersion, which can be used for embedding the emulsion comprising capsaicinoids. Thus it helps to reduce the gastric health hazard by reducing pungency, by means of entrapping the emulsion comprising capsaicinoids, into a solution of gastro-resistant pH dependent polymer by dispersing the emulsion into aqueous solution/dispersion of the polymeric excipient. The polymer also helps to get a non-sticky, free flowing beadlet formulation, although the active is oily, as the emulsion comprising active is embedded within its matrix.

In one embodiment, at least one gastro-resistant pH dependent polymer, as used in the present invention may be selected from sodium alginate, pectin, methacrylate polymers and the combination thereof.

In some embodiments, the modified release formulation is comprised of about 0.5 to 10% by weight of the gastro-resistant pH dependent polymer. More preferably, the formulation may be comprised of about 2 to 7% of at least one gastro-resistant polymer or a combination thereof. According to one more embodiment, the formulation may be comprised of at least one gastro-resistant pH dependent polymer or more than one polymers in combination thereof.

It will be appreciated that the gastro-resistant pH dependent polymer, used for embedding the emulsion comprising capsaicinoids can be interchangeable with the modified release agent used as a coating in the formulation of the present invention. However, function of the polymer which is used in the core and coating is different. As per important embodiment of the invention, at least one gastro-resistant pH dependent polymer used in the active core entraps capsaicinoids in the form of matrix and may reduce or conceal pungency of such active, thus avoiding the irritation to the formulator or operator, during the preparation of the formulation.

The terminology ‘release modifying agent’ as used herein relates to the excipient employed in the coating of the formulation, which helps to release the active at specific pH conditions in gastrointestinal tract. Further, the release modifying agents are sometimes also known as enteric coating polymers, which are pH sensitive polymers that are insoluble in the acidic pH of stomach, but only dissolve and expose the active core in alkaline pH of the intestine, thus allowing complete release and absorption of the active in intestine and avoiding the irritation in large intestine or rectal area due to residual unabsorbed pungent capsaicinoids. The release modifying agent avoids the release of encapsulated active in the core in the stomach, thus protecting gastric mucosa from possible irritation which may be caused due to release of capsaicinoids in the stomach.

According to the important embodiment of the invention, the release modifying agent of the present invention may be selected from hydroxypropyl methylcellulose phthalate, hydroxypropyl methylcellulose acetate succinate, hydroxypropyl methylcellulose, sodium alginate, pectin methacrylate, acetate succinate, polyvinyl acetate phthalate, cellulose acetate phthalate, cellulose acetate trimellitate, carboxymethylethylcellulose, acrylic acid polymers and co-polymers and methacrylic acid polymers and co-polymers, polymethacrylic acid methyl methacrylate and polymethacrylic acid ethyl methacrylate, shellac and the combination thereof.

As per one embodiment of the present invention, the modified release formulation may be comprised of at least one release modifying agent or the combination thereof. The release modifying agent is used in the form of coating, therefore it can be also called as functional coating excipient of the formulation. The release modifying agent as used herein, also acts as a functional enteric coat; wherein the coating facilitates release of high dosage of the pungent active substance in the intestine to reduce irritation and avoid or reduce the abdominal pain and gastric discomfort associated with its release in the stomach.

According to one important embodiment of the invention, the formulation comprising capsaicinoids may be comprised of about 15 to 50% by weight of at least one release modifying agent or the combination thereof. In some embodiments, the formulation may preferably be comprised of about 15 to 50% by weight of release modifying agent or the combination thereof.

As per one more embodiment, the modified release formulation as described herein may be comprised of at least one or more layers of coatings, comprised of at least one release modifying agent or the combination thereof. The coating applied on the active core may alternatively be called as functional coating, as it has important role in release of the active from the formulation.

According to one embodiment the functional coating, as mentioned herein, may employ at least one release modifying agent.

As per one more embodiment, the functional coating may be comprised of two or more release modifying agents in combination to prevent release of active in the stomach.

The terminology ‘processing aid’ as used herein relates to the excipients, which are employed to facilitate the process for preparation of the formulation and these excipients are acceptable in pharmaceutical, nutraceutical and food industry. These excipients are commonly known and used in unit formulation processes such as coating, compression, drying, capsule filling and so on, and also carry out different functions.

The modified release formulation comprising capsaicinoids, may be further converted in final dosage forms such as suitable compressible dosage form, can be filled in capsules or used in the form of sachets as dry mix for beverage, dry syrups, or may be formulated as liquid syrup, health drink, diet drink, and can also be administered through fruit juices, soft drinks and the like, for convenient administration to the consumers.

For the purpose of the present invention, the processing aids may be selected from the group of, but not limited to non-pareil seeds, diluents, binder, coating agents, odour masking agent, glidants, lubricants, plasticizers and the combination thereof. The processing aids may be required for facilitating the conversion of the modified release of the present invention in final dosage forms.

The non-pareil seeds (also synonymously called as core) as used herein may be selected from the material having acidic, basic as well as neutral properties, such as tartaric acid, lactose, microcrystalline cellulose and cellulose derivatives, starch, sucrose, mannitol, sugar, isomalt, xylitol, anhydrous dibasic calcium phosphate, calcium carbonate, silica, and the combination thereof.

According to one embodiment of the present invention, the modified release formulation comprising capsaicinoids, as described herein may employ non-pareil seeds in the range of 5% to 50% w/w of the composition. Preferably, non-pareil seeds containing tartaric acid may be used, as per main embodiment of the present invention. The oily active comprising capsaicinoids can be deposited on non-pareil seeds, in the form of emulsion embedded in aqueous solution or dispersion of gastro-resistant pH dependent polymer, to get active core.

As per one embodiment of the invention, other processing aids such as lubricants, glidants and plasticizers may be employed while depositing the active on non-pareil seeds.

These may be selected from talc, starch, magnesium stearate, silicon dioxide, glycerol monostearate emulsion, glycerine, triethyl citrate and mixtures thereof. The processing aid employed to prevent the agglomeration during process may vary from 2 to 20% by weight of the total mass.

The odour masking agent used may be selected from but not limited to menthol, peppermint oil, lime oil and/or combination thereof. This processing aid is useful to mask pungent odour of the capsaicinoids, while preparation of emulsion and entrapping in the matrix of gastro-resistant pH dependent polymer, thus reducing irritation to the operators and the formulators.

According to further embodiment of the present invention, the modified release formulation may be comprised of about 1 to 10% by weight of odour masking agent.

In some embodiment, odour masking agent may be used in effort to reduce the manufacturing health hazards by reducing pungent odour evolving during emulsion preparation of the active, which can be achieved by adding or dissolving flavouring agents such as menthol, peppermint oil, lime oil in capsicum oleoresin before heating. In some embodiments, the composition contains a weight percentage (w/w) at a range of at or about 1 to 10%.

The formulation may be comprised of diluents known in the art, but not limited to microcrystalline cellulose, silicified microcrystalline, powdered cellulose, microfine cellulose, corn starch, rice bran extract, mannitol, maltodextrin, calcium phosphate, dibasic calcium phosphate, tribasic calcium phosphate, calcium sulphate, or mixtures thereof. The diluents may also be selected from glucose, lactose, sucrose, dextrose, fructose, compressible sugar, or mixtures thereof.

The binders may be selected from the group of low viscosity cellulose derivatives such as hydroxypropylmethyl cellulose (HPMC), hydroxypropyl cellulose (HPC), ethylcellulose, carboxymethylcellulose (CMC), sodium CMC, potassium CMC, calcium CMC, methylcellulose, hydroxyethyl cellulose (HEC), microcrystalline cellulose; polyvinylpyrrolidone (PVP), vinyl pyrrolidone-vinyl acetate copolymer, polyvinyl alcohol, starch, gums like xanthan gum, guar gum, acacia, locust bean gum, alginates, or mixtures thereof.

In one more embodiment, the pH modifier may be selected from the group such as, but not limited to citric acid, trisodium citrate, lactic acid, tartaric acid, L-arginine, calcium carbonate, magnesium carbonate, and the combination thereof.

In one more embodiment, the antioxidant may be selected from the group such as, but not limited to natural tocopherols, ascorbyl palmitate, rosemary extract, epigallocatechin gallate, catechins, ascorbic acid and the combination thereof.

The modified release composition, as described herein, is comprised of
a) active core comprising capsaicinoids, at least one emulsifier and at least one gastro-resistant pH dependent polymer,
b) a functional coating comprising at least one release modifying agent or the combination thereof; wherein the coating facilitates release of capsaicinoids in the intestine to reduce irritation and minimize abdominal pain and gastric discomfort.
c) an optional outermost coat; wherein the coating facilitates oral administration of the formulation in the consumers by avoiding exposure to disagreeable pungent odour and sensory characteristics of capsaicinoids at sufficiently high dose, when using the formulation in the form of dry syrup, health drinks, diet drinks, fruit juices, soft drinks, and also while mixing it with fruit juices or other liquid vehicles for convenient administration to the consumers.

The process for preparation of the modified release formulation, as described herein, may be comprised of emulsification of capsaicinoids using at least one emulsifier or the combination thereof, obtained from natural, synthetic or semi-synthetic source, having varying HLB values.

The o/w type of emulsion obtained in the present invention is a thermodynamically stable fluid that differs from kinetically stable regular o/w type of emulsions, which will separate into oil and water over time. The observed particle size of emulsion, as described herein, measured using Zetasizer Nano ZS, version 7.13, Malvern instrument, may range from about 10-1000 nm, preferably 100-800 nm and more preferably 200 to 500 nm with the observed zeta potential is −50 to −55 mV. The emulsion obtained as per the invention described herein has a very good stability.

The emulsion obtained may be mixed with aqueous solution of at least one gastro-resistant pH sensitive polymer, wherein the emulsion is entrapped in the matrix of the polymer. The emulsion may be then deposited on non-pareil seed to get the active core, comprising capsaicinoids, emulsifier and at least one gastro-resistant pH sensitive polymer. The active core may be coated with at least one release modifying agent, followed by optional outermost coating to get the modified release beadlet formulation comprising capsaicinoids. The formulation, as described herein does not give the pungent taste when it is converted or used in other dosage forms such as dry syrup, health drinks, diet drinks, fruit juices, soft drinks, and the like and therefore it is useful for administration to the consumers as a sports nutrition.

It will be appreciated that the release modifying agent and a gastro-resistant pH dependent polymer used as an entrapment polymer can be interchangeable within the scope of this invention. Accordingly, in a given composition, the gastro-resistant entrapment polymer used in the active core and a release modifying agent used in functional coating can be same or different from those used as entrapping polymers.

In some embodiments, efforts are directed to reduce the gastric health hazards by reducing pungency, which can be achieved by entrapping the emulsion comprising capsaicinoids in a solution of at least one pH dependent gastro-resistant polymer by dispersing the emulsion into aqueous solution/dispersion of the polymer.

The process as described herein, for preparation of modified release capsaicinoid formulation, is free of solvents and employs commonly used industrial equipment, such as mixer, blender, homogenizer for preparation of emulsion; while the fluid bead coater is used for depositing the emulsion matrix on the non-pareil seed and also for coating the active core with at least one release modifying excipient and optionally the outermost coating.

The process for preparation of modified release formulation comprising capsaicinoids is simple, environment friendly and cost effective. The process of the invention is also designed in such a way so that the irritation to the formulator or the operator, while working on the formulation is minimised. The processing aid such as menthol, as used herein, may facilitate to mask the pungent odour of capsaicinoids, during preparation of the formulation. Use of at least one gastro-resistant pH dependent polymer also entraps the capsaicinoids emulsion, in order to minimise the pungent odour or other sensory characteristics of the active.

The formulation as described herein aims to prevent release of capsaicinoids in gastric region and achieve complete intestinal absorption of capsaicinoids in the intestine, thus eliminate the discomfort which may be caused by residual unabsorbed active in gastrointestinal tract. The formulation also aims to avoid pungent taste when the formulation is mixed with dosage forms such as dry syrup, health drinks, diet drinks, fruit juices, soft drinks and the like, thus enabling use of the formulation of the invention for various applications in the consumers as a sports nutrition.

The invention as described herein relates to the modified release formulation, comprising capsaicinoids and process for preparation, wherein emulsion comprising capsaicinoids is entrapped in at least one gastro-resistant polymeric excipient and deposited on non-pareil seed to get active core, which may be further coated using at least one release modifying agent to get the capsaicinoids beadlets. The beadlet formulation, as described herein, may range in size from 100 to 1000 microns.

The modified release formulation, as described herein, is evaluated for its dissolution profile in simulated acidic as well as alkaline conditions, to understand the behaviour of the formulation in gastrointestinal tract. The formulation as described herein, releases not more than 10% by weight of capsaicinoids in stomach within 2 hours, followed by complete release of not less than 75% by weight of capsaicinoids in the intestine within 1 hour.

The formulation is also checked for release of active in the acidic buffers of various pH conditions, in order to check suitability of the beadlet formulation for beverage applications in the form of dry mix, diet mix or also for adding in the fruit juices and suitable liquid vehicles for convenient oral administration to the consumers.

The active substances like capsaicinoids may exhibit higher solubility in ethanolic solutions as compared to water. Concomitant consumption of alcoholic beverages with such products may possibly induce dose dumping in stomach which may lead to issues like gastric irritation, burning and ulceration. The modified release formulation comprising capsaicinoids should be robust with respect to the presence of alcohol in the consumer. Therefore, the formulation of the invention is also checked for the effect of concomitant consumption of alcoholic beverages on dose dumping of capsaicinoids in stomach. Testing for alcohol induced dose dumping is performed using the same dissolution medium (0.1N HCL) with 0, 5, 10 and 20% ethanol added therein, and the release of active is observed to be unaffected due to presence of added ethanol in the dissolution medium, thus confirming the robust nature of the formulation of the invention for the effect of alcohol induced dose dumping.

The modified release formulation, as described herein, eliminates gastric discomfort and facilitates complete release of the active ingredient in the intestine, thereby minimizing the discomfort caused by residual unabsorbed active ingredient in stomach and also ensures complete intestinal absorption of the active. The formulation may also be further coated by the outermost coating, which may reduce the possibility of exposure of the consumers to the pungent taste or odor, and related sensory characteristics of administration of such formulation, even at sufficiently high dosages, thus enhancing the compliance for long term use. The formulation, as described herein can be administered safely to the subjects as a sports nutrition, for enhancing the exercise capacity and endurance and for weight management.

### Experimental Data:

The present invention is now illustrated by means of non-limiting examples.

### Example 1: Modified Release Capsaicinoids Formulation of Formula 1, 2 and 3

TABLE 1
Composition of Modified Release Capsaicinoids formulation

| | | Formula 1 | Formula 2 | Formula 3 |
|---|---|---|---|---|
| Sr. No | Ingredients | (% w/w) | (% w/w) | (% w/w) |
| 1 | Capsaicin oleoresin | 20.00 | 35.00 | 29.00 |
| 2 | Microcrystalline cellulose | 54.00 | — | — |
| | core | | | |
| 3 | Sugar core | — | 20.50 | — |
| 4 | Tartaric acid core | — | — | 32.50 |
| 5 | Menthol | 2.00 | 3.50 | 3.00 |
| 6 | Glyceryl monostearate | 10.00 | 17.00 | 14.50 |
| 7 | Quillaia concentrate | 2.00 | 3.50 | 3.00 |
| 8 | Sodium alginate | 2.00 | 3.50 | 2.00 |
| 9 | Sodium lauryl sulphate | — | — | 1.50 |
| 10 | Silicon dioxide | 10.00 | 17.00 | 14.50 |
| 11 | Purified water | qs | qs | qs |

| | | | |
|---|---|---|---|
| Total | 100.00 | 100.00 | 100.00 |
| Assay (Total capsaicinoids) | 8.5% | 14.00% | 10% |

### A) Process for Formula 1, 2 and 3:

i. The capsaicin oleoresin is mixed with menthol and about 2 to 5% of glyceryl monostearate. The mixture is melted at temperature of 70 to 80 degree celsius.
ii. Quillaia concentrate is dissolved in water and heated at temperature of 70 to 80 degree celsius
iii. The water is heated separately at temperature of 70 to 80 degree celsius and added in mixture obtained in step-(i) under continue stirring till w/o emulsion is formed.
iv. The step-ii heated solution is added slowly to step-iii emulsion under stirring. The stirring is continued till o/w emulsion formed and then cooled to room temperature.
v. Sodium alginate is dispersed separately in water and added in o/w emulsion obtained in step-iv.
vi. Remaining glyceryl monostearate is used to produce o/w emulsion using quillaia concentrate as emulsifier vii. Glyceryl monostearate emulsion obtained in Step vi is added to step v active entrapped polymer dispersion under stirring.
viii. Silicon dioxide and sodium lauryl sulphate is dispersed in water separately and added to step vii active dispersion under stirring.
ix. Remaining water is added and further stirred for 15 mins to form uniform dispersion.
x. Non-pareil seeds of microcrystalline cellulose or sugar or tartaric acid are loaded in fluid bed processer and the step viii active dispersion is layered on the same by maintaining bed temperature at about 25° C.-35° C.

### B) Dissolution Profile for Formula 1, 2 and 3

Apparatus: USP Type II Paddle, Dissolution medium: 0.1N HCL, 750 ml, RPM: 100, Temperature: 37° C.+/−0.5° C., Time: 2 Hrs, Analysis using HPLC at wavelength 281 nm

TABLE 2
Dissolution data of Modified Release Capsaicinoids formulation

| | % Release of formulation | | |
|---|---|---|---|
| Time (hr) | Formula 1 | Formula 2 | Formula 3 |
| 1 hr | 3% | 14% | 32% |
| 2 hr | 6% | 16% | 38% |

The dissolution data of Table 2 indicates that use of at least one gastro-resistant pH dependent polymer, such as sodium alginate, avoids release of significant amount of entrapped active (capsaicinoids) from active core in acidic medium in 2 hours. Dissolution profile of Formula 3 also indicates that, even though sodium lauryl sulphate is present in the active core, the release of active in acidic pH is insignificant because of use of at least one gastro-resistant pH dependent polymer in the formulation. The irritation due to release of pungent active is thus minimized due to optimized selection of at least one gastro-resistant pH dependent polymer in the active core of the formulation.

### Example 02: Modified Release Capsaicinoids Formulation of Formula 4, 5 and 6

TABLE 03
Composition of Modified Release Capsaicinoids formulation

| | | | | |
|---|---|---|---|---|
| | | Formula 4 | Formula 5 | Formula 6 |
| Sr. No | Ingredients | (% w/w) | (% w/w) | (% w/w) |

Active Core

| | | | | |
|---|---|---|---|---|
| 1 | Capsaicin oleoresin | 7 | 32.5 | 18.3 |
| 2 | Microcrystalline | 19.4 | 13.6 | — |
| | cellulose core | | | |
| 3 | Tartaric acid core | — | — | 11.3 |
| 4 | Menthol | 0.7 | 1.6 | 1.8 |
| 5 | Glyceryl monostearate | 3.5 | 5 | 9.2 |
| 6 | Quillaia concentrate | 0.7 | 2.4 | 1.8 |
| 7 | Sodium alginate | 0.07 | 3.2 | 2.8 |
| 8 | Silicon dioxide | 3.5 | 12.2 | 9.2 |
| 9 | Sodium lauryl sulphate | — | — | 0.5 |
| 10 | Purified water | qs | Qs | qs |

Modified release coat

| | | | | |
|---|---|---|---|---|
| 11 | Sodium alginate | 37.8 | — | 3.8 |
| 12 | Pectin | 6.7 | — | — |
| 13 | Anionic methacrylate | — | 23.5 | 31.6 |
| | polymer | | | |
| 14 | Glycerine | 8.9 | — | 0.8 |
| 15 | Glyceryl monostearate | 11.1 | 6 | 8.9 |
| | emulsion | | | |
| 16 | Purified water | qs | Qs | qs |

| Total | 100 | 100 | 100 |
|---|---|---|---|
| Particle Size | 400 to | 250 to | 250 to |
| | 850 | 600 | 600 |
| | microns | microns | microns |
| Assay (Total capsaicinoids) | 2.96% | 5.87% | 7.51% |

### A) Process for Formula 4, 5 and 6

The process for preparation of composition is defined in the stepwise manner as follows:
i. Active core is prepared as per the examples 01
ii. Sodium alginate and/or pectin and/or anionic methacrylate polymer is dispersed in water.
iii. Further glycerine is added to dispersion obtained in step-ii (Formula 4 and 6)
iv. The Glyceryl monostearate emulsion is added to step iii solution (Formula 4 and 6) OR step ii solution (Formula 5)
v. Remaining water is added and further stirred for 15 mins to form uniform dispersion.
vi. Active core is loaded in Fluid bed processer and the step v functional coating dispersion is layered on the same by maintaining bed temperature about 25° C.-35° C.

### C) Dissolution Profile for Formula 4, 5 and 6

Apparatus: USP Type II Paddle, Dissolution medium: 0.1N HCL (750 ml), pH 6.8 with 0.5% SLS (1000 ml), RPM: 100, Temperature: 37° C.+/−0.5° C., Time: 2 Hrs, Analysis using HPLC at wavelength 281 nm

TABLE 04
Dissolution data of Modified Release Capsaicinoids formulation

| | | | |
|---|---|---|---|
| Time (hr) | Formula 4 | Formula 5 | Formula 6 |

% Release in 750 mL of 0.1N HCL

| | | | |
|---|---|---|---|
| 2 hr | 8% | 0% | 2% |

% Release in 1000 mL of pH 6.8 with 0.5% SLS

| | | | |
|---|---|---|---|
| 1 hr | 91% | 88% | 100% |

The modified release formulation comprising capsaicinoids, as described herein, releases less than 10% of capsaicinoids in simulated gastric fluid in 2 hours. The formulation releases more than 85% of capaicinoids within 1 hour, in simulated intestine condition, at pH 6.8.

### Dose Dumping Study—for Formula 4

### Apparatus: USP Type II Paddle,

### Dissolution Medium:

0.1N HCL (750 ml) containing 0% ethanol,
0.1N HCL (750 ml) containing 5% ethanol,
0.1N HCL (750 ml) containing 10% ethanol,
0.1N HCL (750 ml) containing 20% ethanol,
RPM: 100, temperature: 37° C.+/−0.5° C., Time: 2 Hrs, analysis using HPLC at wavelength 281 nm

TABLE 05
Alcohol Dose dumping study of Formula 4

| | 750 ml 0.1N | 750 ml 0.1N | 750 ml 0.1N | 750 ml 0.1N |
|---|---|---|---|---|
| | HCl contains | HCl contains | HCl contains | HCl contains |
| | 0% ethanol | 5% ethanol | 10% ethanol | 20% ethanol |
| Time (hr) | % Release in respective Media | | | |
| 1 hr | 4% | 8% | 6% | 4% |
| 2 hr | 8% | 12% | 12% | 8% |

The formulation of the invention is tested for alcohol induced dose dumping study using the same dissolution medium (0.1N HCL) with 0, 5, 10 and 20% ethanol for 2 hours. It was observed that presence of 5 to 20% of ethanol in the dissolution medium has no impact on release profile of the formulation, thus the formulation is robust for effect of alcohol in the stomach and alcohol induced dose dumping is not observed for the formulation of the invention, thus avoiding the adverse effects such as irritation, discomfort or ulceration due to release of pungent active, such as capsaicinoids in the stomach in presence of alcohol.

### Example 03: Modified Release Capsaicinoids Formulation of Formula 7, 8 and 9

TABLE 07
Composition of Modified Release Capsaicinoids formulation

| | | | | |
|---|---|---|---|---|
| | | Formula 7 | Formula 8 | Formula 9 |
| Sr. No | Ingredients | (% w/w) | (% w/w) | (% w/w) |

Active Core

| | | | | |
|---|---|---|---|---|
| 1 | Capsaicin oleoresin | 21.1 | 18.7 | 17.3 |
| 2 | Sugar core | 12.3 | 10.9 | — |
| 3 | Tartaric acid core | — | — | 15.8 |
| 4 | Menthol | 2.1 | 1.9 | 1.7 |
| 5 | Glyceryl monostearate | 10.60 | 9.30 | 6.90 |
| 6 | Quillaia concentrate | 2.10 | 1.90 | 1.70 |
| 7 | Sodium alginate | 2.10 | 1.90 | 1.70 |
| 8 | Silicon dioxide | 10.60 | 9.30 | 6.90 |
| 9 | Purified water | Qs | qs | qs |

Modified release coat

| | | | | |
|---|---|---|---|---|
| 10 | Sodium alginate | 16.70 | 14.80 | 4.90 |
| 11 | Pectin | 3.00 | 2.60 | — |
| 12 | Anionic methacrylate | 8.40 | — | 32.90 |
| | polymer | | | |
| 13 | Shellac | — | 14.50 | — |
| 14 | Glycerine | 3.90 | 6.40 | 1.00 |
| 15 | Glyceryl monostearate | 7.00 | 7.90 | 9.20 |
| | emulsion | | | |
| 16 | Purified water | Qs | qs | qs |

| Total weight | 100 | 100 | 100 |
|---|---|---|---|
| Particle Size | 400 to | 400 to | 150 to |
| | 850 | 850 | 250 |
| | microns | microns | microns |
| Assay (Total capsaicinoids) | 8.3% | 7.4% | 6.4% |

### Process:

The process as described in Example 2 is followed to prepare capsaicinoids formulation of formula 7, 8 and 9

### B) Dissolution Profile for Formula 7, 8 and 9:

Apparatus: USP Type II Paddle, Dissolution medium: 0.1N HCL (750 ml), pH 6.8 with 0.5% SLS (1000 ml), RPM: 100, Temperature: 37° C.+/−0.5° C., Time: 2 Hrs, Analysis using HPLC at wavelength 281 nm

TABLE 08
Dissolution data of Modified Release Capsaicinoids formulation

| | | | |
|---|---|---|---|
| Time (hr) | Formula 7 | Formula 8 | Formula 9 |

% Release in 750 mL of 0.1N HCL

| | | | |
|---|---|---|---|
| 2 hr | 1% | 0% | 1% |

% Release in 1000 mL of pH 6.8 with 0.5% SLS

| | | | |
|---|---|---|---|
| 1 hr | 102% | 88% | 92% |

The formulations exhibit minimum release (about 1%) of capsaicinoids in acidic pH (stomach conditions) in 2 hours; while more than 85% of capsaicinoids is released in alkaline pH (intestine condition) within 1 hour, thus indicating immediate and complete release from the formulation, ensuring effective absorption of the active, thus avoiding any irritation in the colon or rectal area due to residual unabsorbed capsaicinoids.

### Example 04: Modified Release Capsaicinoids Formulation

TABLE 08
Composition of Modified Release Capsaicinoids formulation

| | | Formula 06 | Formula 10 |
|---|---|---|---|
| Sr. No | Ingredient | (% w/w) | (% w/w) |
| 1 | Formula 06 | 100 | 83.3 |
| 2 | Basic methacrylate polymer | — | 12.1 |
| 3 | Stearic acid | — | 1.2 |
| 4 | Tartaric acid | — | 1.2 |
| 5 | Glyceryl monostearate emulsion | — | 2.2 |
| 6 | Purified water | — | q.s |

| | | |
|---|---|---|
| Total | 100 | 100 |
| Assay Total (Capsaicinoids) | 7.51% | 6.1% |

### Process:

The process as described in Example 1 is followed to prepare Capsaicinoids formulation of formula 3.

The 83.3% w/w of formula 3 is coated with basic methacrylate polymer using the processing aids such as stearic acid, tartaric acid, glyceryl monostearate emulsion to form beadlet. The beadlets are collected and sifted.

The beadlets are then tested for release profile in various dissolution media such as 0.1 N HCl, pH 6.8 buffer, normal water and citrate buffers of varying pH such as 3, 4 and 5, to check the effect of outermost coating on the beadlets.

B) Dissolution Profile: The dissolution profile was evaluated using apparatus USP Type II Paddle, dissolution medium 0.1N HCL (750 ml) for 2 hrs, followed by pH 6.8 buffer with 0.5% SLS (1000 ml) for 1 hr at 100 rpm and temperature 37° C.+/−0.5° C., using HPLC at wavelength 281 nm.

TABLE 09
Dissolution data of Modified Release Capsaicinoids formulation

| | | |
|---|---|---|
| Time (hr) | Formula 6 | Formula 10 |

% Release in 750 mL of 0.1N HCL

| | | |
|---|---|---|
| 2 hr | 2% | 1% |

% Release in 1000 mL of pH 6.8 with 0.5% SLS

| | | |
|---|---|---|
| 1 hr | 100% | 95% |

Release in 300 mL of drinking water, pH 7

| | | |
|---|---|---|
| 1 hr | 0.05 mg | No Capsaicinoids detected |
| 2 hr | 0.11 mg | No Capsaicinoids detected |

Release in 300 mL of citrate buffer pH 3.0

| | | |
|---|---|---|
| 1 hr | 0.08 mg | No Capsaicinoids detected |
| 2 hr | 0.14 mg | No Capsaicinoids detected |

Release in 300 mL of citrate buffer pH 4.0

| | | |
|---|---|---|
| 1 hr | 0.06 mg | No Capsaicinoids detected |
| 2 hr | 0.13 mg | No Capsaicinoids detected |

Release in 300 mL of citrate buffer pH 5.0

| | | |
|---|---|---|
| 1 hr | 0.07 mg | No Capsaicinoids detected |
| 2 hr | 0.11 mg | No Capsaicinoids detected |

It was observed that, when the optional outermost coat is applied on the beadlets prepared by Formula 6, the coated formula 10 does not release the active in various pH conditions such as pH 3, 4, 5 and 7, as seen in the above table (quantity of capsaicinoids was non-detectable), as compared to the release of active from Formula 6, which is not coated with any outermost coating of basic methacrylate polymer.

This indicates that the outermost coating facilitates consumption of the formulation of the invention, without being seriously exposed to the associated disagreeable or uncomfortable odors, aromas, and sensory characteristics of such material at sufficiently high dosages, especially when mixed with other formulations such as dry syrup, health drinks, diet drinks, fruit juices, soft drinks, and the like.

### Example 05: Compression of Modified Beadlets

TABLE 10
Composition of Modified Release Capsaicinoids formulation

| Sr. No | Ingredient | Formula 11 | | Formula 12 | |
|---|---|---|---|---|---|
| 1 | Modified Release Pellets | 27 | mg | — | |
| | as per Formula 6 | | | | |
| 2 | Modified Release Pellets | — | | 34 | mg |
| | as per formula 4 | | | | |
| 3 | Microcrystalline cellulose | 121.8 | mg | 114.8 | mg |
| 4 | Croscarmellose sodium | 4.8 | mg | 4.8 | mg |
| 5 | Colloidal silicon dioxide | 3.2 | mg | 3.2 | mg |
| 6 | Magnesium stearate | 3.2 | mg | 3.2 | mg |

| | | | | |
|---|---|---|---|---|
| Total | 160 | mg | 160 | mg |
| Assay Total (Capsaicinoids) | 2 | mg | 2 | mg |

### Process:

The process as described in Example 2 is followed to prepare capsaicinoids formulation of formula 4 and 6.

The 27 mg of formula 6 and 34 mg of formula 4 are taken separately and further added with processing aids such as microcrystalline cellulose, croscarmellose sodium, colloidal silicon dioxide, magnesium stearate. The lubricated blend was compressed with 8 mm round punch into compressed mass having 2 mg total capsaicinoids.

### B) Dissolution Profile:

Apparatus: USP Type II Paddle, Dissolution medium: 0.1N HCL (750 ml), pH 6.8 with 0.5% SLS (1000 ml), RPM: 100, Temperature: 37° C.+/−0.5° C., Time: 2 Hrs, Analysis using HPLC at wavelength 281 nm

TABLE 11
Dissolution data of Modified Release Capsaicinoids formulation

| | | |
|---|---|---|
| Time (hr) | Formula 11 | Formula 12 |

Release in 750 mL of 0.1N HCL

| | | |
|---|---|---|
| 2 hr | 2 | 0 |

Followed by

% Release in 1000 mL of pH 6.8 with 0.5% SLS

| | | |
|---|---|---|
| 1 hr | 96% | 87% |

The tablet formulations exhibited about 2% of active release in 0.1 N HCl in 2 hours and immediate release of more than 85% of capsaicinoids in alkaline pH of the intestine. Thus the formulation exhibits modified release of capsaicinoids, even after conversion in compressible dosage form like tablets.

### Example 06: Evaluation of the Cross-Section Morphologies by Scanning Electron Microscope

The cross-section morphologies are visualized using Scanning Electron Microscope (Quanta 200, Field Electron and Ion Company, USA). The samples are mounted on an aluminium stub using double sided tape and the beadlets are placed upside down on butter paper with its core faced downwards, and the beadlets are stuck on the stub. Air blown on the stub using the rubber blower to eliminate free particles. XT microscope control software is used to run the samples. The photographs are taken at an acceleration voltage of 20 kV.

### I. Sample—As Per Formula 5

In FIG. 1, scanning electron microscopy (SEM) photographs show two layers for beadlets prepared by depositing the emulsion containing active entrapped/embedded in at least one gastro-resistant pH dependent polymers, which is further coated with at least one release modifying agent or the combination thereof. The non-pareil seed (shown as 1) is of microcrystalline cellulose, active layering (Shown as 2) is of emulsion containing capsaicinoids embedded in at least one gastro-resistant pH dependent polymer layered on Microcrystalline cellulose, and functional coating (shown as 3) is of Anionic methacrylate polymer coating.

### II. Sample—As Per Formula 7

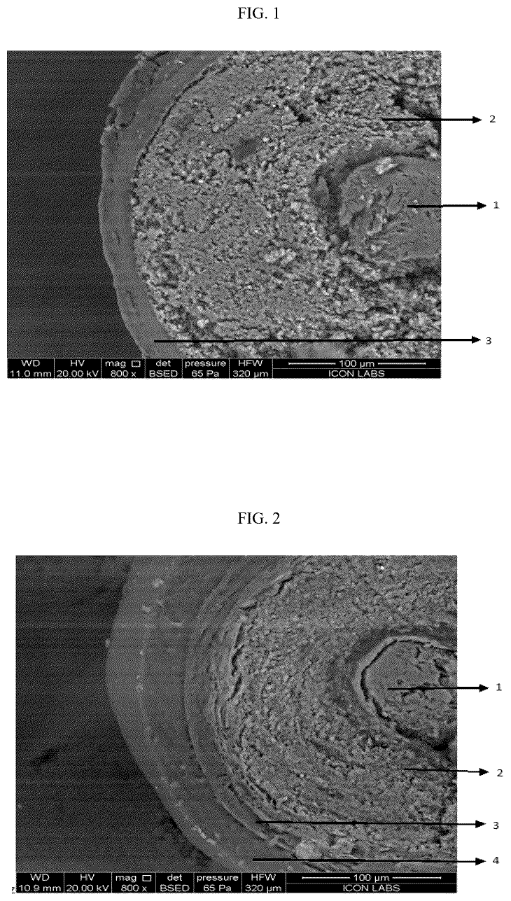

In FIG. 2, scanning electron microscopy (SEM) photographs show three layers for beadlets prepared by depositing the emulsion containing active entrapped/embedded in at least one gastro-resistant pH dependent polymer, which is further coated with at least one release modifying agent. The non-pareil seed of Sugar (shown as 1), active layering of emulsion containing capsaicinoids embedded in at least one gastro-resistant pH dependent polymer layered on Sugar (shown as 2), coating of Sodium alginate polymer (shown as 3) and functional coating of anionic methacrylate polymer (as shown 4).

### III. Sample—As Per Formula 10

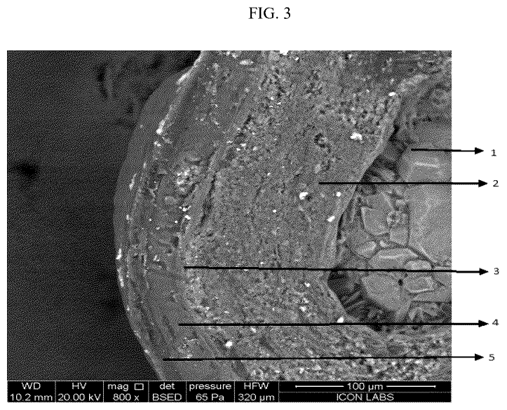

In FIG. 3, scanning electron microscopy (SEM) photographs show four layers for beadlets prepared by depositing the emulsion containing active entrapped/embedded in at least one gastro-resistant pH dependent polymer, which is coated by using at least one release modifying agent, followed by optional outermost layer. The non-pareil seed of tartaric acid, active layering of emulsion containing capsaicinoids embedded in at least one gastro-resistant pH dependent polymer layered on Tartaric acid (shown as 2), layer of Sodium alginate polymer (shown as 3), functional coating of anionic methacrylate polymer (as shown 4) and optional outermost coat of basic methacrylate polymer (as shown 5).

We claim:

1. A modified release formulation, comprising;
a. an active core comprising capsaicinoids, at least one emulsifier and at least one gastro-resistant pH dependent polymer or the combination thereof;
b. a functional coating comprising at least one release modifying agent or the combination thereof, and
c. at least one processing aid, acceptable in pharmaceutical, nutraceutical and food industry;
wherein the formulation facilitates complete release of capsaicinoids in the intestine to avoid discomfort in the gastro-intestinal tract.

2. The modified release formulation as claimed in claim 1, comprising
0.5 to 20% by weight of capsaicinoids,
0.5 to 10% by weight of emulsifiers,
0.5 to 10% by weight of at least one gastro-resistant pH dependent polymer,
15 to 50% by weight of at least one release modifying agent.

3. The modified release formulation as claimed in claim 1, which may be further comprised of an optional outermost coating selected from the group of methyl cellulose, agar, sodium alginate, polyvinyl pyrrolidone, cellulose polymers, starch, gum arabic, xanthan gum, polyethylene glycol and the combination thereof.

4. The modified release formulation as claimed in claim 1, comprised of combination of emulsifiers, selected from natural, synthetic and semisynthetic source, with varying HLB values.

5. The modified release formulation as claimed in claim 4, wherein the emulsifier from synthetic source may be selected from potassium laurate, triethanolamine stearate, sodium lauryl sulfate, alkyl polyoxyethylene sulfates, sodium dodecyl sulfate, dioctyl sodium sulfosuccinate, cetyltrimethyl ammonium bromide, lauryldimethyl benzylammonium chloride, polyoxyethylene fatty alcohol ethers, Sorbitan fatty acid esters, Polyoxyethylene alkyl ethers (macrogols) Polyoxyethylene sorbitan fatty acid esters, Polyoxyethylene polyoxypropylene block copolymers (poloxamers), Polyethylene glycol 400 monostearate, lanolin alcohols, ethoxylated lanolin and the combination thereof.

6. The modified release formulation as claimed in claim 4, wherein the emulsifier from natural source may be selected from quillaia concentrate, acacia, tragacanth, alginates, chondrus, xanthan, gelatin, egg yolk, casein, wool fat, cholesterol, wax, lecithin and the combination thereof.

7. The modified release formulation as claimed in claim 4, wherein the emulsifier from semi-synthetic source may be selected from methyl cellulose, carboxymethyl cellulose, sodium carboxymethyl cellulose and the combination thereof.

8. The modified release formulation as claimed in claim 4, wherein at least one gastro-resistant pH dependent polymer is selected from sodium alginate, pectin, methacrylate polymers and the combination thereof.

9. The modified release formulation as claimed in claim 1, wherein at least one release modifying polymeric excipient is selected from hydroxypropyl methylcellulose phthalate, hydroxypropylmethylcellulose acetate succinate, hydroxypropyl methylcellulose, sodium alginate, pectin methacrylate, acetate succinate, polyvinyl acetate phthalate, cellulose acetate phthalate, cellulose acetate trimellitate, carboxymethyl ethylcellulose, acrylic acid polymers and co-polymers, methacrylic acid polymers and co-polymers, polymethacrylic acid methyl methacrylate and polymethacrylic acid ethyl methacrylate, shellac and the combination thereof.

10. The modified release formulation as claimed in claim 1, wherein at least one processing aid may be selected from the group comprising of non-pareil seeds, fillers, diluents, lubricants, binders, glidants, odour masking agent, anti-caking agents, channelizing agents, vehicles, buffers, acidifiers, alkalizers, complexing agents, gum bases, antioxidants, viscosity enhancers, and the combination thereof.

11. The modified release formulation as claimed in claim 10, wherein the non-pareil seeds may be selected from the material having acidic, basic as well as neutral properties, such as tartaric acid, lactose, microcrystalline cellulose and cellulose derivatives, starch, sucrose, mannitol, sugar, isomalt, xylitol, anhydrous dibasic calcium phosphate, calcium carbonate, silica, and the combination thereof.

12. The modified release formulation as claimed in claim 1, which exhibits release of not more than 10% by weight of capsaicinoids in stomach within 2 hours, followed by release of not less than 75% by weight of capsaicinoids in intestine within 1 hour; and does not exhibit alcohol induced dose dumping effect in the stomach.

13. A process for preparation of modified release formulation, which is comprised of
i) mixing capsaicinoids with 0.5 to 10% by weight of combination of emulsifiers selected from natural, synthetic or semi-synthetic sources, to get a stable emulsion
ii) entrapping the emulsion with at least 0.5 to 10% by weight of at least one gastro-resistant polymer
iii) depositing the mixture of step (ii) on non-pareil seeds to get active core
iv) coating the active core obtained after step iii) with 15 to 50% by weight of at least one release modifying agent to get modified release capsaicinoid beadlets, having the size in range of 100 to 1000 microns.
v) optionally coating the modified release capsaicinoid beadlets with outermost coating to facilitate masking the taste and odour of the active for administration in the form of dry mix for beverage and related forms.

14. The process of modified release formulation as claimed in claim 14, which is solvent-free and does not make use of any organic solvent in the preparation of active core and the coating in the modified release formulation.

15. The modified release formulation as claimed in claim 1, which can be converted in suitable compressible dosage form, can be filled in capsules or used in the form of sachets for use as dry mix for beverage, dry syrups, or may be formulated as liquid syrup, health drink, diet drink, and can also be administered through fruit juices, soft drinks and the like, for convenient administration to the consumers.
